(19) United States
(12) Patent Application Publication
(10) Pub. No.: US 2023/0372651 A1
(43) Pub. Date: Nov. 23, 2023

(54) VENTILATOR-TO-VENTILATOR TRANSFER OF VENTILATOR DATA

(71) Applicant: COVIDIEN LP, Mansfield, MA (US)

(72) Inventors: Patrick J. Nellis, Georgetown (CA); Mark Y. Su, Boulder, CO (US); Kiran Shankaranarayana, San Diego, CA (US); Balaji Murugan, Carlsbad, CA (US)

(73) Assignee: COVIDIEN LP, Mansfield, MA (US)

(21) Appl. No.: 18/319,120

(22) Filed: May 17, 2023

Related U.S. Application Data
(60) Provisional application No. 63/344,802, filed on May 23, 2022.

(51) Int. Cl. A61M 16/00; G16H 40/63

(52) U.S. Cl. CPC A61M 16/024; G16H 40/63; A61M 2205/6018; A61M 2205/502

(57) ABSTRACT

Systems and methods for transfer of ventilator settings from one ventilator to another ventilator are disclosed. a method for transferring ventilator settings from one medical ventilator to another medical ventilator. An example method includes receiving a selection to initiate a transfer of current ventilator settings to a receiving ventilator, the current ventilator settings corresponding to settings currently being used by the medical ventilator to provide ventilation to a patient; exchanging identification data with the receiving ventilator; based on the exchanged identification data, establishing communication with the receiving ventilator; and prior to the patient being disconnected from the medical ventilator, transmitting the current ventilator settings to the receiving ventilator.

### CROSS-REFERENCE TO RELATED APPLICATIONS

This application claims the benefit of U.S. Provisional Application No. 63/344,802 filed May 23, 2022, entitled “Ventilator-to-Ventilator Transfer of Ventilator Data,” which is incorporated herein by reference in its entirety.

### INTRODUCTION

Medical ventilator systems have long been used to provide ventilatory and supplemental oxygen support to patients. These ventilators typically comprise a connection for pressurized gas (air, oxygen) that is delivered to the patient through a conduit or tubing. As each patient may require a different ventilation strategy, modern ventilators may be customized for the particular needs of an individual patient. For example, several different ventilator modes or settings have been created to provide better ventilation for patients in different scenarios, such as mandatory ventilation modes, spontaneous ventilation modes, and assist-control ventilation modes.

It is with respect to this general technical environment that aspects of the present technology disclosed herein have been contemplated. Furthermore, although a general environment is discussed, it should be understood that the examples described herein should not be limited to the general environment identified herein.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Among other things, aspects of the present disclosure include systems and methods for transfer of ventilator data from one medical ventilator to another medical ventilator. In an aspect, the technology relates to a method for transferring ventilator settings from one medical ventilator to another medical ventilator. The method includes receiving a selection to initiate a transfer of current ventilator settings to a receiving ventilator, the current ventilator settings corresponding to settings currently being used by the medical ventilator to provide ventilation to a patient; exchanging identification data with the receiving ventilator; based on the exchanged identification data, establishing communication with the receiving ventilator; and prior to the patient being disconnected from the medical ventilator, transmitting the current ventilator settings to the receiving ventilator.

In an example, the method further includes receiving a selection of a subset of the current ventilator settings for transfer to the receiving ventilator; and transmitting the current ventilator settings includes transmitting the selected subset of the current ventilator settings. In another example, the method further includes displaying a transfer settings interface including a setting-selection menu and a transfer activation element; receiving, via the setting-selection menu, a selection of a subset of ventilator settings for transfer to the receiving ventilator; receiving a selection of the transfer activation element; and based on receiving the selection of the transfer activation element, initiating the transmission, wherein transmitting the ventilator settings includes transmitting the selected subset of ventilator settings. In yet another example, the method further includes receiving an indication of a ventilator type of the receiving ventilator; based on the ventilator type of the receiving ventilator and the current ventilator settings, generating recommended ventilator settings; and wherein the transmitted current ventilator settings are the recommended ventilator settings. In a further example, the indication of the ventilator type is based on input received via a user interface presented by the medical ventilator. In another further example, the indication of the ventilator type is based on identification data received from the receiving ventilator. In still another example, the method further includes transmitting, to the receiving ventilator, patient data for the patient.

In another aspect, the technology relates to a medical ventilator for transferring ventilator settings. The ventilator includes a display; a processor; and memory storing instructions that, when executed by the processor, cause the medical ventilator to perform a set of operations. The operations include delivering ventilation to a patient based on current ventilator settings; receiving a selection to initiate a transfer of the current ventilator settings to a receiving ventilator; exchanging identification data with the receiving ventilator; based on the exchanged identification data, establishing communication with the receiving ventilator; and prior to the patient being disconnected from the medical ventilator, transmitting the current ventilator settings to the receiving ventilator.

In an example, the operations further include receiving a selection of a subset of the current ventilator settings for transfer to the receiving ventilator; and transmitting the current ventilator settings includes transmitting selected subset of the current ventilator settings. In another example, the operations further include displaying, on the display, a transfer settings interface including a setting-selection menu and a transfer activation element; receiving, via the setting-selection menu, a selection of a subset of ventilator settings for transfer to the receiving ventilator; receiving a selection of the transfer activation element; and based on receiving the selection of the transfer activation element, initiating the transmission, wherein transmitting the ventilator settings includes transmitting the selected subset of ventilator settings. In yet another example, the operations further include receiving an indication of a ventilator type of the receiving ventilator; based on the ventilator type of the receiving ventilator and the current ventilator settings, generating recommended ventilator settings; and wherein the transmitted current ventilator settings are the recommended ventilator settings. In a further example, the indication of the ventilator type is based on input received via a user interface presented by the medical ventilator. In another further example, the indication of the ventilator type is based on identification data received from the receiving ventilator. In still another example, the operations further include transmitting, to the receiving ventilator, patient data for the patient.

In another aspect, the technology relates to a method for receiving ventilator settings from a transferring ventilator by a receiving ventilator. The method includes exchanging identification data with the transferring ventilator; based on the exchanged identification data, establishing communication with the transferring ventilator; prior to a patient being connected to the receiving ventilator, receiving, by the receiving ventilator, ventilator settings from the transferring ventilator; and delivering ventilation to the patient, by the receiving ventilator, according to the received ventilator settings.

In an example, the method further includes displaying, by the receiving ventilator prior to the patient being connected, the received ventilator settings. In another example, the method further includes receiving a confirmation approval of the received ventilator settings, and wherein delivering ventilation is commenced based on receiving the confirmation approval. In yet another example, the method further includes receiving a selection to change the received ventilator settings; displaying an interface to change the ventilator settings; and receiving, via the interface, changes to the ventilator settings. In still another example, the method further includes receiving, from the transferring ventilator, patient data for the patient being ventilated on the transferring ventilator. In a further example, the method also includes based on the received patient data, accessing previously stored ventilator settings for the patient.

It is to be understood that both the foregoing general description and the following Detailed Description are explanatory and are intended to provide further aspects and examples of the disclosure as claimed.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 depicts a diagram illustrating an example of a medical ventilator connected to a human patient.

FIG. 2 depicts a data exchange between two medical ventilators.

FIG. 3 depicts an example time series data exchange between two medical ventilators.

FIG. 4A depicts an example user interface displayed a medical ventilator for transferring settings.

FIG. 4B depicts another example user interface displayed on a medical ventilator for transferring settings.

FIG. 4C depicts another example user interface displayed on a medical ventilator for transferring settings.

FIG. 4D depicts an example user interface displayed on a medical ventilator receiving transferred ventilator settings.

FIG. 4E depicts another example user interface displayed on a medical ventilator receiving transferred ventilator settings.

FIG. 4F depicts another example user interface displayed on a medical ventilator for transferring settings.

FIG. 5 depicts a flowchart illustrating an example method for transferring ventilator settings.

FIG. 6 depicts a flowchart illustrating an example method for transferring ventilator settings.

FIG. 7 depicts a flowchart illustrating an example method for receiving ventilator settings.

FIG. 8 depicts a method for associated ventilators with one another.

FIG. 9 depicts another method for associating ventilators with one another.

FIG. 10 depicts another method for transferring data from one ventilator to another ventilator.

While examples of the disclosure are amenable to various modifications and alternative forms, specific aspects have been shown by way of example in the drawings and are described in detail below. The intention is not to limit the scope of the disclosure to the particular aspects described. On the contrary, the disclosure is intended to cover all modifications, equivalents, and alternatives falling within the scope of the disclosure and the appended claims.

### DETAILED DESCRIPTION

As discussed briefly above, medical ventilators are used to provide breathing gases to patients who are otherwise unable to breathe sufficiently. In modern medical facilities, pressurized air and oxygen sources are often available from wall outlets, tanks, or other sources of pressurized gases. Accordingly, ventilators may provide pressure regulating valves (or regulators) connected to centralized sources of pressurized air and pressurized oxygen. The regulating valves function to regulate flow so that respiratory gases having a desired concentration are supplied to the patient at desired pressures and flow rates. Further, as each patient may require a different ventilation strategy, modern ventilators may be customized for the particular needs of an individual patient.

In some cases, a patient needs to be transferred from one medical ventilator to another medical ventilator. For example, when a patient is undergoing surgery or being transported from one location to another location, the patient may be transferred from a first ventilator to a second ventilator. In these cases, the first ventilator and the second ventilator may be of a different type. For instance, the first ventilator may be a bedside ventilator and may be a more fully featured ventilator with greater capabilities than the second ventilator, which may be a transport ventilator. Accordingly, the available settings and modes, along with performance, between the two ventilators may differ. Providing similar ventilation to the patient by both ventilators, however, may be desired. Currently, a clinician needs to have deep knowledge of both ventilator types, the patient's condition, and/or the settings currently being implemented in the first ventilator in order to manually enter these settings into the second ventilator. This process may be prone to error and also requires significant time from the clinician, which could be spent tending to the care of other patients. Additional scenarios also exist where a patient may need to be transferred from one ventilator to another. For example, when a patient returns to the bedside, the patient is transferred from the transport ventilator to the bedside ventilator. In other examples, a failure of one ventilator may require the patient to transferred to another ventilator. A worsening condition of a patient may also cause the patient to need to be transferred to a more fully featured ventilator. In each of these cases, the receiving ventilator requires substantial configuration before receiving the patient.

Among other things, the present technology addresses the above problems by providing for ventilators that are capable of transferring ventilator settings and other data (e.g., patient data) between ventilators. For instance, a clinician may initiate a transfer option on the first ventilator. Activation of the transfer option may cause the ventilator settings to be directly transferred to the second ventilator. In other examples, one or more graphical user interfaces (GUIs) may be presented to facilitate the transfer process. The GUIs may include a GUI for selecting the ventilator settings and/or additional data that is to be transferred to the second ventilator. In some examples, a GUI may be displayed to provide for a selection of the type of the second ventilator type. Based on the selected second ventilator type, the first ventilator may then determine if the current ventilator settings and ventilator mode are available on the second ventilator. If the corresponding settings and mode are not available, or there would be ventilator performance differences even with the same mode and settings, the first ventilator may generate a closest mode and closest or recommended settings to be transferred to the second ventilator. A clinician may then review the recommended mode and/or settings to approve or change the settings and/or mode to be transferred. Once approved, the ventilator settings and/or mode are transferred to the second ventilator. As such, upon the patient being connected to the second ventilator, the second ventilator may immediately begin delivering substantially the same ventilation to the patient that was being delivered by the first ventilator.

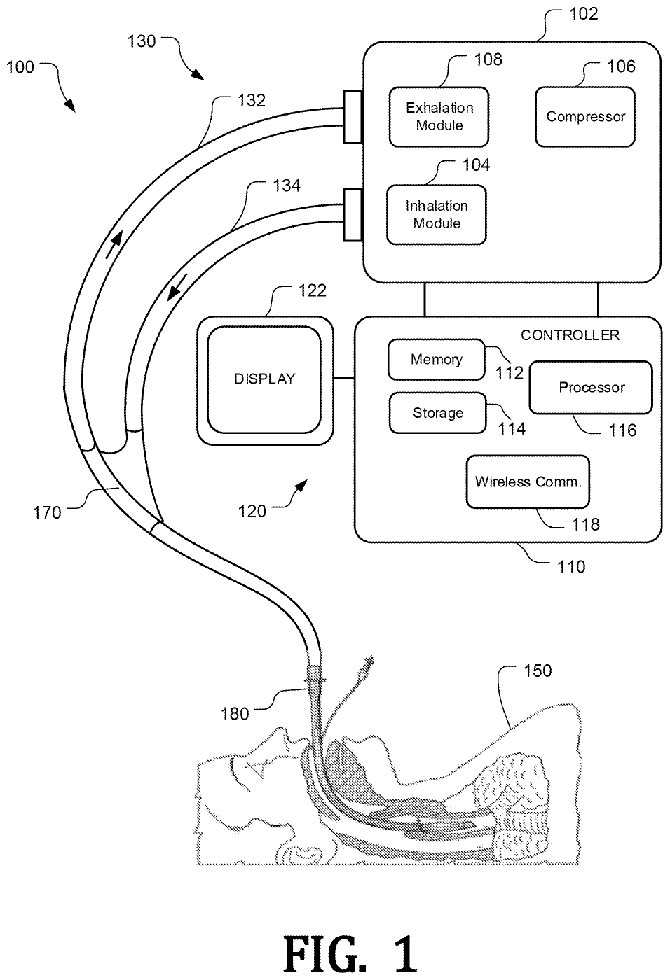

FIG. 1 is a diagram illustrating an example of a medical ventilator 100 connected to a human patient 150. The ventilator 100 may provide positive pressure ventilation to the patient 150. Ventilator 100 includes a pneumatic system 102 (also referred to as a pressure generating system 102) for circulating breathing gases to and from patient 150 via the ventilation tubing system 130, which couples the patient to the pneumatic system via an invasive (e.g., endotracheal tube, as shown) or a non-invasive (e.g., nasal mask) patient interface.

Ventilation tubing system 130 may be a two-limb (shown) or a one-limb circuit for carrying gases to and from the patient 150. In a two-limb example, a fitting, typically referred to as a “wye-fitting” 170, may be provided to couple a patient interface 180 to an inhalation limb 134 and an exhalation limb 132 of the ventilation tubing system 130.

Pneumatic system 102 may have a variety of configurations. In the present example, system 102 includes an exhalation module 108 coupled with the exhalation limb 132 and an inhalation module 104 coupled with the inhalation limb 134. Compressor 106 or other source(s) of pressurized gases (e.g., air, oxygen, and/or helium) is coupled with inhalation module 104 to provide a gas source for ventilatory support via inhalation limb 134. The pneumatic system 102 may include a variety of other components, including mixing modules, valves, sensors, tubing, accumulators, filters, etc., which may be internal or external sensors to the ventilator (and may be communicatively coupled, or capable communicating, with the ventilator).

Controller 110 is operatively coupled with pneumatic system 102, signal measurement and acquisition systems, and an operator interface 120 that may enable an operator to interact with the ventilator 100 (e.g., change ventilator settings, select operational modes, view monitored parameters, etc.). Controller 110 may include memory 112, one or more processors 116, storage 114, and/or other components of the type found in command and control computing devices. In the depicted example, operator interface 120 includes a display 122 that may be touch-sensitive and/or voice-activated, enabling the display 122 to serve both as an input and output device.

For the purposes of this disclosure, a “breath” refers to a single cycle of inspiration and exhalation delivered with the assistance of a ventilator. The term “breath type” refers to some specific definition or set of rules dictating how the pressure and flow of respiratory gas are controlled by the ventilator during a breath. For example, breath types may be mandatory mode breath types where the initiation and termination of the breath is made by the ventilator, or spontaneous mode breath types where the breath is initiated (and in some cases also terminated) by the patient. Examples of spontaneous breath types include proportional assist (PA) breath type, volume support (VS) breath type, pressure support (PS) breath type, etc. Examples of mandatory breath types include a volume control breath type, a pressure control breath type, volume-targeted pressure control breath type, etc.

A ventilation “mode,” on the other hand, is a set of rules controlling how multiple subsequent breaths should be delivered. A simple mandatory mode of ventilation is to deliver one breath of a specified mandatory breath type at a clinician-selected respiratory rate, f (e.g., one breath every 6 seconds). A single ventilation mode may mix different breath types, delivering a first breath type and then in a subsequent breath delivering a second, different breath type, according to the rules of the mode. Typically, ventilators will continue to provide breaths of the specified breath type(s) as dictated by the rules defining the mode, until the mode is changed by a clinician.

The memory 112 includes non-transitory, computer-readable storage media that stores software that is executed by the processor 116 and which controls the operation of the ventilator 100. In an example, the memory 112 includes one or more solid-state storage devices such as flash memory chips. In an alternative example, the memory 112 may be mass storage connected to the processor 116 through a mass storage controller (not shown) and a communications bus (not shown). Although the description of computer-readable media contained herein refers to a solid-state storage, the computer-readable storage media may be any available media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid-state memory technology, CD-ROM, DVD, or other optical storage, magnetic cassettes, magnetic tape, magnetic disk storage or other magnetic storage devices, or any other medium which can be used to store the desired information and which can be accessed by the computer.

Communication between the ventilator 100 may be accomplished via wireless communication circuitry 118 of the ventilator 100. The wireless communication circuitry 118 may include receivers, transmitters, and/or transceivers capable of transmitting and/or receiving data wirelessly. For instance, the wireless communication circuitry 118 may include components for communicating over short-range or direct wireless communication protocols, such as BLUETOOTH, BLUETOOTH LOW ENERGY, infrared, radio-frequency identification (RFID), ZIGBEE, WI-FI DIRECT, LI-FI, etc. In other examples, local area networks (LANs) and/or wide area networks (WANs) may be used to wireless transfer the ventilator settings. For example, the present technology may use a layer built over the TCP/IP protocol. TCP/IP stands for “Transmission Control Protocol/Internet Protocol” and provides a basic communication language for many local networks (such as intra- or extranets) and is the primary communication language for the Internet. Specifically, TCP/IP is a bi-layer protocol that allows for the transmission of data over a network. The higher layer, or TCP layer, divides a message into smaller packets, which are reassembled by a receiving TCP layer into the original message. The lower layer, or IP layer, handles addressing and routing of packets so that they are properly received at a destination. In some examples, the data may also be transferred or transmitted between a wired connection between two ventilators.

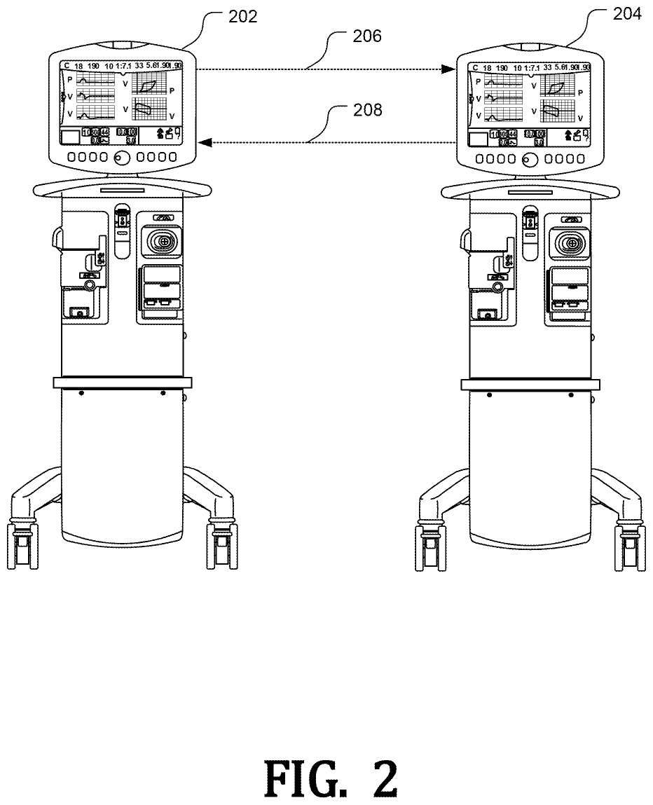

FIG. 2 depicts a data exchange between two medical ventilators, including a first ventilator 202 and a second ventilator 204. The first ventilator 202 transfers or transmits data 206 to the second ventilator 204. The second ventilator 204 may also transfer or transmit data 208 to the first ventilator 202.

The data 206, 208 exchanged between the first ventilator 202 and the second ventilator 204 may include a variety of different information. For example, the data 206, 208 may include pairing data that is used to establish wireless communication between the first ventilator 202 and the second ventilator 204. Such data may include identifying information about the respective ventilators 202, 204. For example, the data 206, 208 may include packets of data that are used by link control and manager protocols as well as service discovery protocols. Such packets may include authentication, encryption, and other data used to establish the communication link between the first ventilator 202 and the second ventilator 204.

Once the communication link (e.g., BLUETOOTH pairing) between the first ventilator 202 and the second ventilator 204 is established, additional data 206, 208 may be exchanged. For instance, in an example where a patient is being ventilated on the first ventilator 202 and is to be transferred to the second ventilator 204, the data 206 may include ventilator settings for the ventilation currently being delivered to the patient. The ventilator settings may include data regarding the ventilation mode and the ventilator settings corresponding to the ventilation mode. Example ventilation modes include modes such as volume assist/control, pressure assist/control, pressure support ventilation, volume synchronized intermittent mandatory ventilation (SIMV), and pressure SIMV, BiLevel, among others. Ventilator settings may include settings that may be changed by the operator or clinician, such as tidal volume, respiratory rate, inspiratory time, inspiratory pressure, triggering sensitivity, cycling sensitivity, I:E ratio, positive end-expiratory pressure (PEEP), fraction of inspired oxygen (FiO₂), among others.

The data 206 may also include demographic information about the patient, such as the sex, height, weight, age, etc. of the patient. In addition, in some examples, the data 206 may also include recent ventilatory and/or medical data about the patient for a recent preceding time period, such as the last 30 minutes or hour that the patient has been on the first ventilator 202. Such ventilator data (e.g., pressure waveforms, flow waveforms, volume waveforms) may be viewed on the second ventilator 204 once such ventilatory data is received by the second ventilator 204. For instance, monitored patient data may be transferred, which is based on measured data of the ventilation being provided to the patient. Accordingly, a clinician is able to review prior ventilatory data for the patient on the second ventilator 204 even when the ventilatory data was generated on the first ventilator 202.

The data 208 provided by the second ventilator 204 to the first ventilator 202 may also indicate the type of ventilator of the second ventilator 204. The type of ventilator 204 may include a brand, model, or other identifying information of the second ventilator 204. Additionally or alternatively, the data 208 provided by the second ventilator 204 to the first ventilator 202 may include capability data for the second ventilator 204. The capability data may indicate the types of modes and/or breath types, along with the corresponding ventilator settings, that the second ventilator 204 is able to provide. The first ventilator 202 may then use that information to determine what ventilator settings should be transferred to the second ventilator 204. As such, the data transferred between ventilators may be include patient data, monitored patient data, settings data, alarms data, trend data, waveform data, device configuration data, device state or status data, user configurations, among other types of data.

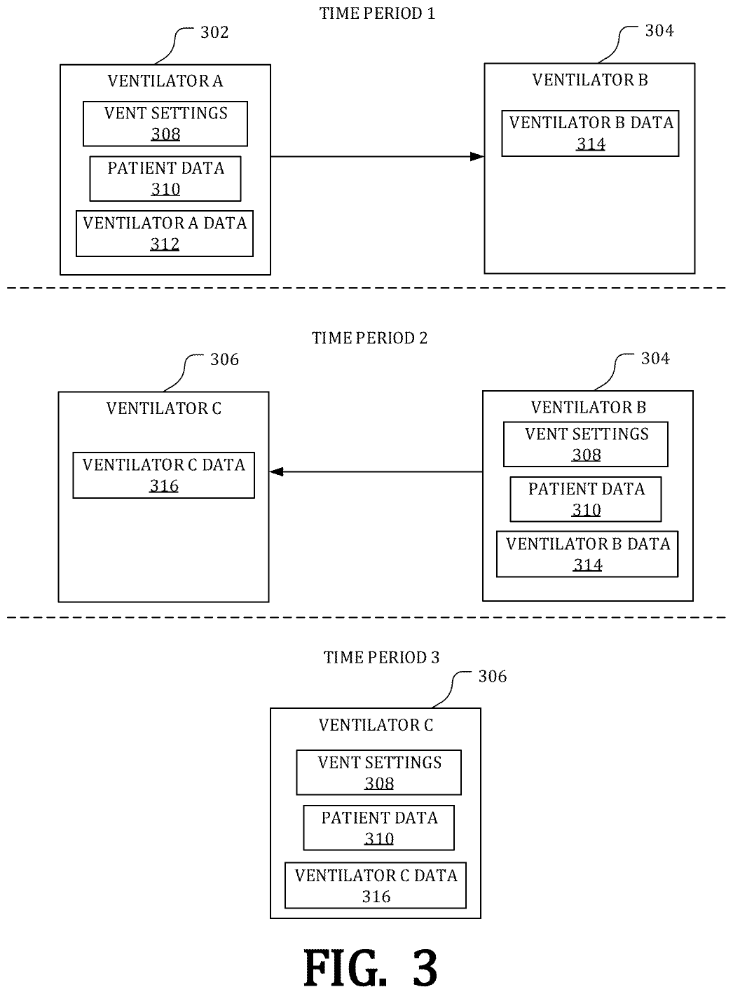

FIG. 3 depicts an example time series data exchange between three medical ventilators, including a first ventilator 302 (“Ventilator A”), a second ventilator 304 (“Ventilator B”), and a third ventilator 306 (“Ventilator C”). During a first time period, a patient is currently ventilated by the first ventilator 302 and is being transferred to the second ventilator 304. The first ventilator 302 is ventilating the patient according to ventilator settings 308. The first ventilator 302 also stores patient data 310 about the patient, and first ventilator identification data 312 for the first ventilator 302. The patient data 310 may include demographic information about the patient, such as the sex, height, weight, age, etc. of the patient. The first ventilator identification data 312 may include brand, model, and/or other identifying information (e.g., media access control address (MAC) address, internet protocol (IP) address, etc.) to identify the first ventilator 302.

Prior to the patient being transferred to the second ventilator 304, the first ventilator 202 may transfer one or more of the ventilator settings 308 and/or the patient data 310 to the second ventilator 304. Prior to receiving the ventilator settings 308, the second ventilator 304 includes its own ventilator identification data 314 for the second ventilator 304. The second ventilator identification data 314 may include brand, model, and/or other identifying information (e.g., media access control address (MAC) address, internet protocol (IP) address, etc.) to identify the second ventilator 304. In some examples, the second ventilator 304 may send the second ventilator identification data 314 to the first ventilator 302 to facilitate the types of settings that are transmitted from the first ventilator 302 to the second ventilator 304. In addition, the first ventilator 302 and the second ventilator 304 may exchange the first ventilator identification data 312 and the second ventilator identification data 314 to establish a wireless connection for transfer of the settings over the wireless connection.

At a second time period after the first time period, the ventilator settings 308 and the patient data 310 have been transferred to the second ventilator 304. During the second time period, the second ventilator 304 is ventilating the patient according to the ventilator settings 308. During the second time period, the patient is to be transferred from the second ventilator 304 to the third ventilator 306. Accordingly, the second ventilator 304 may establish a wireless connection with the third ventilator 306 by exchanging the second ventilator identification data 314 and third ventilator identification data 316 of the third ventilator 306.

Once the wireless communication is established between the second ventilator 304 and the third ventilator 306, the second ventilator 304 may transfer the ventilator settings 308 and the patient data 310 to the third ventilator 306. The ventilator settings 308 may be the same settings that were received from the first ventilator 302 in the first time period. In some examples, the second ventilator 304 may modify or adjust the ventilator settings 308 while ventilating the patient. In such examples, the ventilator settings 308 that are transferred from the second ventilator 304 to the third ventilator 306 are the modified ventilator settings 308. In other examples, even where the second ventilator 304 modifies the ventilator settings 308, the second ventilator 304 may store an original version of the ventilator settings 308 that were received from the first ventilator 302. In such examples, the second ventilator 304 may transfer the original version of the ventilator settings 308 to the third ventilator 306.

At a third time period, after the second time period and after the patient has been transferred to the third ventilator 306, the third ventilator 306 delivers ventilation to the patient according to the ventilator settings 308 received from the second ventilator 304. The third ventilator 306 may also store the patient data 310 that was received from the second ventilator 304.

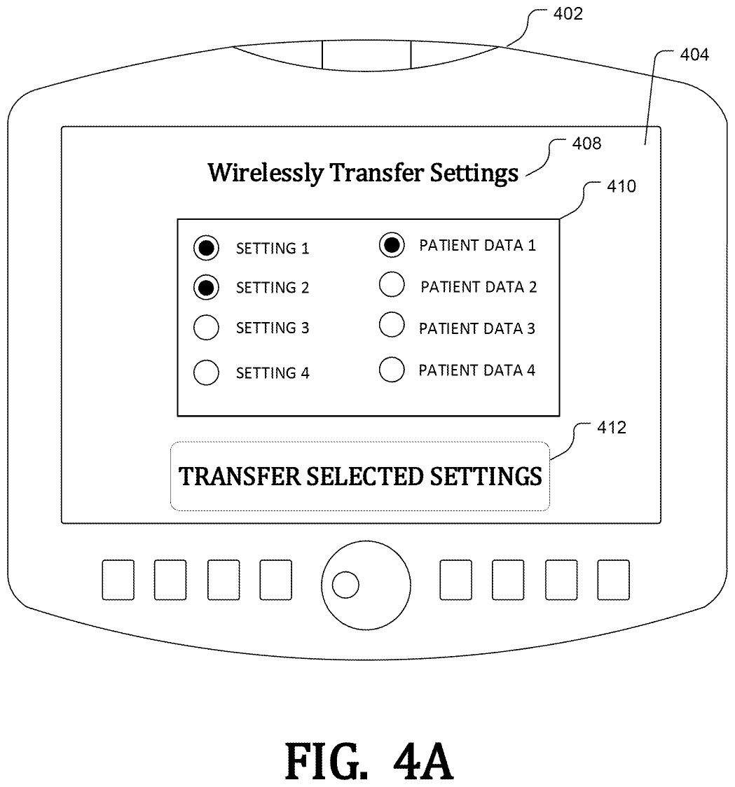

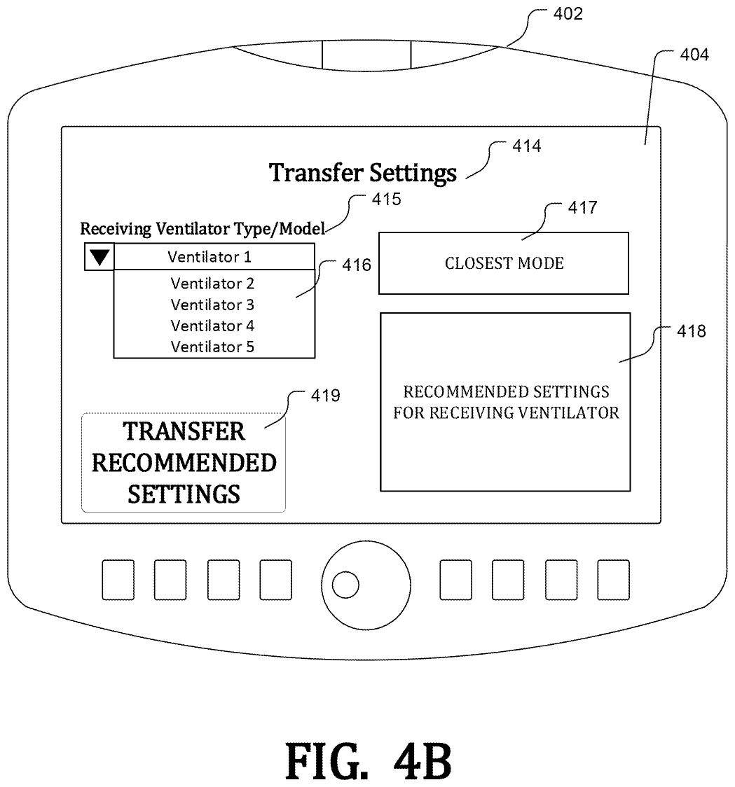

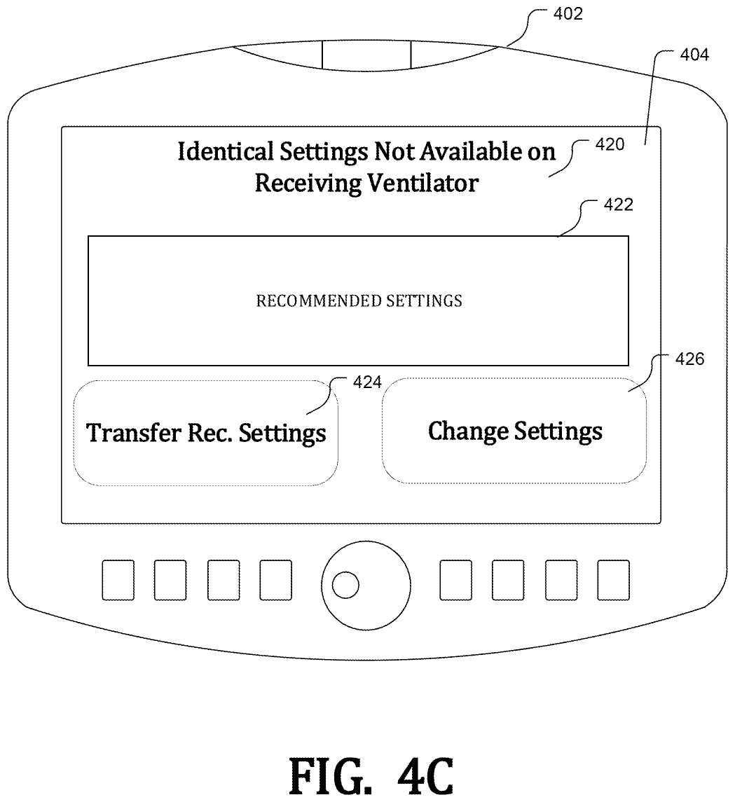

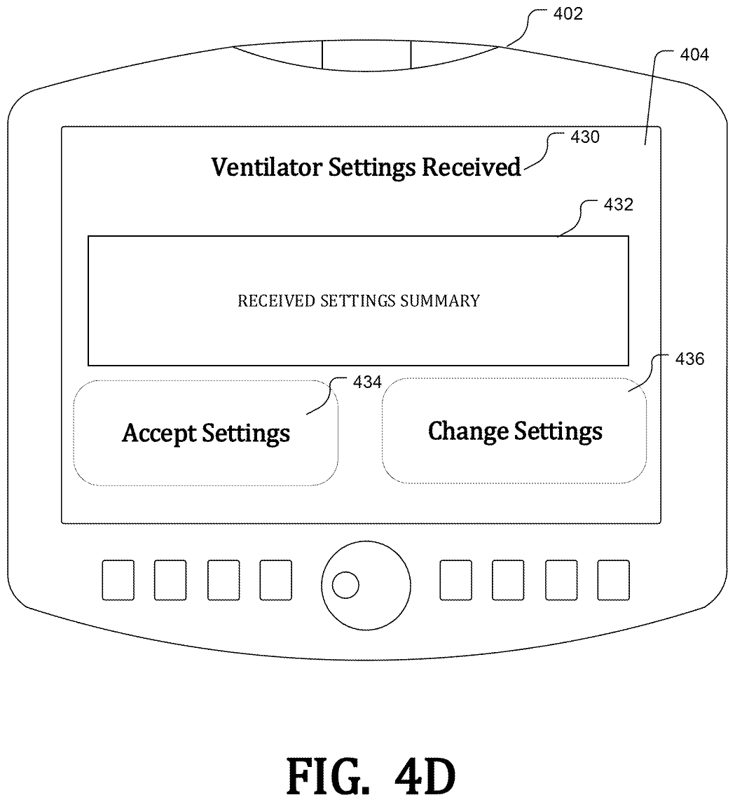

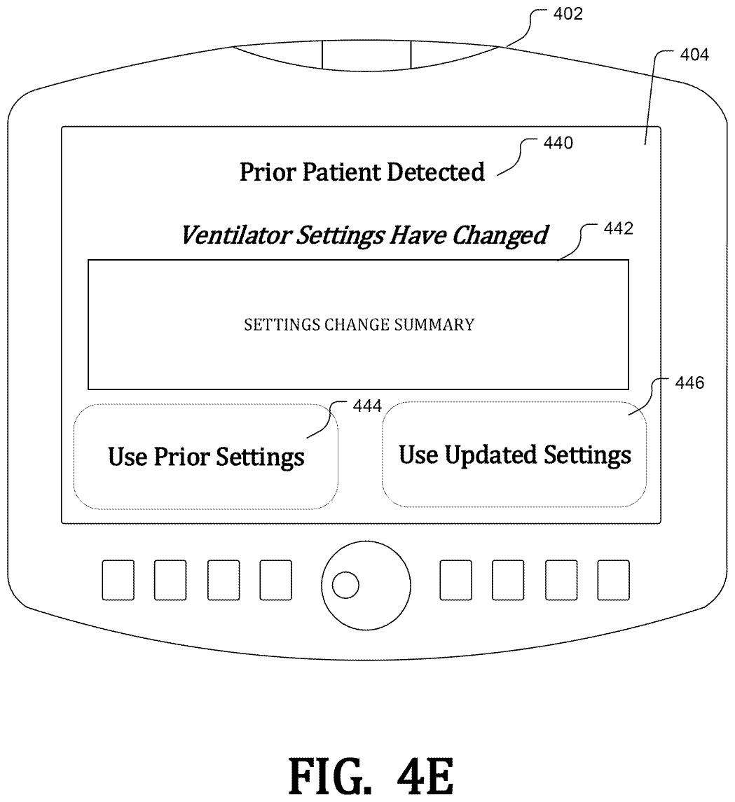

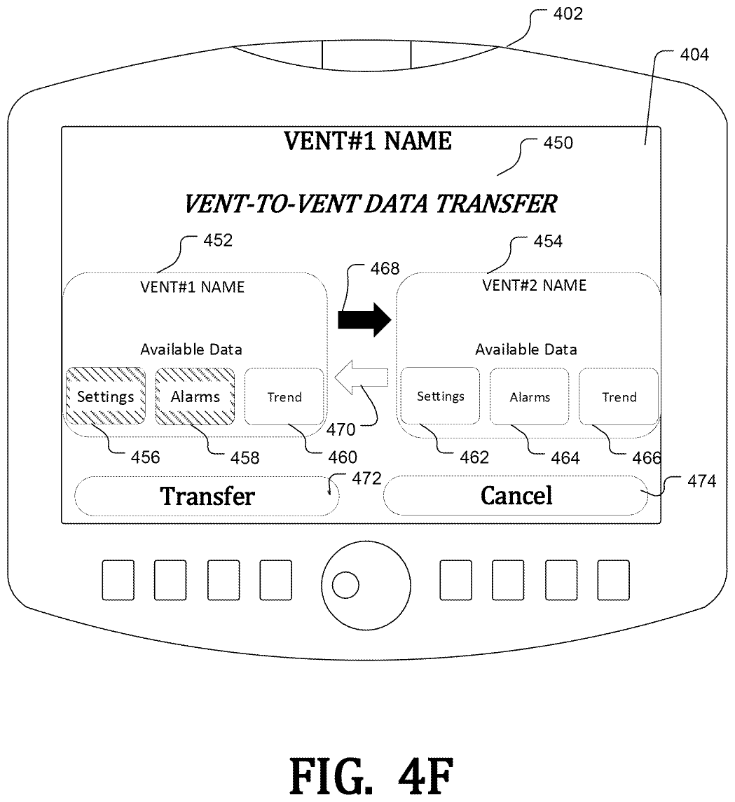

FIG. 4A depicts an example user interface 408 displayed on a medical ventilator for transferring settings. FIG. 4A depicts a display 402 of a medical ventilator and a GUI 404 provided on the display 402. In the example depicted, a transfer settings interface or prompt 408 is displayed in the GUI 404. The transfer settings prompt 408 includes a title indicating that ventilator settings are to be transferred. The transfer settings prompt 408 may be accessed through a selection of an option from a home or main screen provided on the GUI 404 during ventilation of the patient. In some examples, a selectable transfer settings option may be provided in a settings pane provided by the ventilator in the GUI 404, and selection of such an option may cause the display of the prompts and different interfaces discussed herein.

The transfer settings prompt 408 includes a setting-selection menu 410 that provides for selection of ventilator settings that are to be wirelessly transferred to another ventilator. For instance, a plurality of ventilator settings may be displayed for selection by a clinician. In some examples, the current values of the ventilator settings may also be presented in the setting-selection menu 410. While the selection input mechanism displayed in FIG. 4A is a radio button, other types of user interface selection mechanisms may be utilized.

Selectable portions of patient data may also be displayed within the setting-selection menu 410. For example, the clinician may select which portions of the patient data are to be transferred to the second ventilator. The different portions of patient data may correspond to different data items such as sex, height, weight, age, etc. of the patient.

The transfer settings prompt 408 may also include a transfer activation element 412. Once the clinician has selected the ventilator settings and/or patient data in the setting-selection menu 410 that is to be transferred to the second ventilator 304, the clinician may select the transfer activation element 412 to cause the selected ventilator settings and/or patient data to be wirelessly transferred to the second ventilator. Upon receiving a selection of the transfer activation element 412, the ventilator may wirelessly transfer the selected ventilator settings and/or patient data to the second ventilator.

FIG. 4B depicts another example user interface 414 displayed on a medical ventilator for transferring settings. In FIG. 4B, the GUI 404 displays a transfer settings interface or prompt 414 for transferring ventilator settings. The transfer settings prompt 414 includes a receiving ventilator section 415 that allows for a clinician to select the type or model of ventilator that will be receiving the transferred ventilator settings. The receiving ventilator section 415 may include a receiving ventilator selection menu 416 that displays a plurality of different ventilator types that may be selected by the clinician. While depicted as a drop-down box, the receiving ventilator selection menu 416 may take other forms and may be provided via other types of user interface mechanisms.

The transfer settings prompt 414 also includes user interface elements indicating a closest ventilation mode 417 and recommended settings 418. The closest ventilation mode 417 indicates a mode available on the selected receiving ventilator to the current mode of ventilation on the current ventilator. In some examples, the closest ventilation mode 417 may be the same mode of operation as the current mode of operation where such a mode of operation is available on the receiving ventilator. The recommended settings 418 include settings that are closest to, or would provide the closest consistent performance as, the current ventilator settings of the current ventilator. The recommended settings 418 may be the same or different than the current ventilator settings depending on the characteristics and/or characteristics of the receiving ventilator.

The transfer settings prompt 414 also includes a transfer activation element 419. Upon receiving the selection of the transfer activation element 419, the recommended settings 418, which may include the closest ventilation mode 417, are wirelessly transmitted to the receiving ventilator.

When a receiving ventilator type is selected from the receiving ventilator selection menu 416, the current ventilator determines or accesses characteristics and/or capabilities of the receiving ventilator. Based on the characteristics and/or capabilities of the selected receiving ventilator, the current ventilator determines whether the present ventilator settings are available on the receiving ventilator and/or if the present ventilator settings would result in substantially the same performance on the receiving ventilator. Where the same settings are not available on the receiving ventilator, the current ventilator determines the closest ventilation mode 417 and/or recommended settings 418 to provide to closest consistent performance between the current ventilator and the receiving ventilator.

Such determinations relating to generating the closest ventilation mode 417 and the recommended settings 418 may be generated from logic tables and/or translation tables that map the modes and/or ventilator settings of one ventilator to another. The tables may be generated analytically and/or experimentally by running different modes and settings across multiple ventilator types to determine performance similarities and differences. Based on such similarities and differences, the logic tables and/or translation tables may be generated and stored for later access by the ventilator. The tables may be generated beforehand and stored in the memory of the various ventilators.

The transfer settings prompt 414 may be beneficial even where wireless transfer capabilities are unavailable, or fail, on one or more of the current ventilator and/or the receiving ventilator. For example, by determining and displaying the closest ventilation mode 417 and/or the recommended settings 418, the ventilator provides the clinician with the settings that should be used by the receiving ventilator. The clinician may then use that information to manually enter the closest ventilation mode 417 and/or the recommended settings 418 into the receiving ventilator prior to the patient being connected to the receiving ventilator. Accordingly, the current ventilator still helps facilitate the transfer of the patient to the receiving ventilator even where the current ventilator does not or cannot directly transfer the ventilator settings to the receiving ventilator.

FIG. 4C depicts another example user interface 420 displayed on a medical ventilator for transferring settings. In FIG. 4C, the GUI 404 displays another transfer settings prompt 420. The transfer settings prompt 420 is similar to the transfer settings prompt 414 in that the transfer settings prompt 420 provides for recommended settings 422. The recommended settings 422 may be generated in the same manner as the recommended settings 418 in FIG. 4B. The recommended settings 422 may be based on an identification of the receiving ventilator that is automatically performed upon wireless communication being established between the current ventilator and the receiving ventilator. For instance, the current ventilator may receive ventilator identification data from the receiving ventilator. The current ventilator then uses that identification data to determine the capabilities and/or characteristics of the receiving ventilator.

The transfer settings interface or prompt 420 also includes a selectable transfer activation element 424 and a selectable change settings element 426. Upon receiving a selection of the transfer activation element 424, the recommended settings 422 are wireless transmitted to the receiving ventilator by the current ventilator. Upon receiving a selection of the change settings element 426, the current ventilator displays another interface to allow for the clinician to change the ventilator settings and/or values of the settings that are to be transferred to the receiving ventilator. Once the ventilator settings and/or values of the settings are adjusted, the adjusted ventilator settings may be wirelessly transferred to the receiving ventilator.

FIG. 4D depicts an example user interface 430 displayed on a medical ventilator receiving transferred ventilator settings. In FIG. 4D, the GUI 404 includes a received-settings interface or prompt 430. The received-settings prompt 430 may be displayed upon receiving ventilator settings from another ventilator.

The received-settings prompt 430 includes a received-settings summary 432, an accept settings element 434, and a change settings element 436. The received-settings summary 432 provides a summary or listing of the ventilator settings that have been received. The received-settings summary 432 provides a way for a clinician to review the received ventilator settings, and their respective values.

If the clinician agrees with the received ventilator settings, the clinician may select the accept settings element 434. Upon receipt of a selection of the accept settings element 434, the receiving ventilator applies or implements the received ventilator settings such that ventilation of the patient may begin as quickly as possible when the patient is connected to the ventilator.

If the clinician disagrees with the received ventilator settings, the clinician may select the change settings element 436. Upon receiving a selection of the change settings element 436, the receiving ventilator displays another interface to allow for the clinician to change the ventilator settings and/or values of the settings. Once the ventilator settings and/or values of the settings are adjusted, the adjusted ventilator settings may be implemented by the receiving ventilator.

FIG. 4E depicts another example user interface 440 displayed on a medical ventilator receiving transferred ventilator settings. In FIG. 4E, the GUI 404 displays a prior-patient reconnect interface or prompt 440. The prior-patient reconnect prompt 440 is presented when a patient that was previously connected to the current ventilator is to be reconnected to the current ventilator. For instance, a patient may be ventilated by the current ventilator, then transferred to a transport ventilator, then transferred from the transport ventilator back to the current ventilator. In such scenarios, the current ventilator may store the patient data and/or ventilator settings that were used during ventilation of the patient. While the patient is on the transport ventilator, however, a clinician may change the ventilator settings on the transport ventilator. When the current ventilator receives the wirelessly transferred ventilator settings and patient data from the transport ventilator, the current ventilator may compare the stored, prior ventilator settings for the patient with the received ventilator settings. If there are any changes or differences between the stored settings and the received settings, the prior-patient reconnect prompt 440 may be displayed.

The prior-patient reconnect prompt 440 includes a settings change summary 442, a use prior settings element 444, and a use updated settings element 446. The settings change summary 442 provides a summary or indication of the settings that have been changed or adjusted. For instance, the settings change summary 442 may provide a listing of the settings where differences between the stored settings and the received settings differ. The settings change summary 442 thus provides an efficient way for a clinician to analyze the changes or adjustments that occurred while the patient was on the transport ventilator.

If the clinician agrees with the received ventilator settings and the changes indicated in the settings change summary 442, the clinician may selection the use updated settings element 446. Upon receiving the selection of the use updated settings element 446, the received ventilator settings are implemented by the ventilator. If the clinician disagrees with the received ventilator settings and the changes indicated in the settings change summary 442, the clinician may select the use prior settings element 444. Upon receiving the selection of the use prior settings element 444, the ventilator implements the stored, prior ventilator settings for the patient.

FIG. 4F depicts another example user interface displayed on a medical ventilator for transferring settings. In FIG. 4F, the GUI 404 includes transfer-settings interface 450. The transfer settings interface 450 may be accessed through a selection of an option from a home or main screen provided on the GUI 404 during ventilation of the patient. In some examples, a selectable transfer settings option may be provided in a settings pane provided by the ventilator in the GUI 404, and selection of such an option may cause the display of the transfer settings interface.

The transfer settings interface 450 includes a first ventilator representation 452 and a second ventilator representation. For instance, the first ventilator representation 452 may represent the ventilator (e.g., Vent #1) upon which the transfer-settings interface 450 is being displayed. The ventilator representation 454 may represent a second ventilator to which data is to be transferred to or received from. The first ventilator representation 452 includes a plurality of selectable UI elements to indicate the data that is to be transferred from the first ventilator to the second ventilator. For example, the selectable UI elements may include a settings element 456, an alarms element 458, and a trend element 460. When one of the selectable UI elements is selected, the appearance of that element may change to indicate that the element has been selected. For instance, in the example depicted, the settings element 456 and the alarms element 458 have been selected, but the trend element 460 has not been selected. The data corresponding to the selected elements is the data that will be transferred during the data transfer operation. Accordingly, in the example depicted, the settings data and the alarms data of the first ventilator will be transferred.

The second ventilator representation 454 also includes selectable UI elements to indicate data that may be transferred from the second ventilator. The selectable UI elements may be similar or the same as the selectable UI elements in the first ventilator representation 452. For example, the selectable UI elements may include a settings element 462, an alarms element 464, and a trend element 466.

The transfer-settings interface 450 may also include one or more selectable transfer-direction indicators 468, 470, which may be in the form of arrows or other shapes that indicate the direction of the data transfer. A first transfer-direction indicator 468 may be selected to transfer data from the first ventilator to the second ventilator. A second transfer-direction indicator 470 may be selected to transfer data from the second ventilator to the first ventilator. Both the first transfer-direction indicator 468 and the second transfer-direction indicator 470 may be selected to indicate that an exchange of data between the first ventilator and the second ventilator is to occur. The display of the transfer-direction indicators 468, 470 may change when selected. For instance, in the example depicted, the first transfer-direction indicator 468 is selected but the second transfer-direction indicator 470 is not selected.

The example transfer-settings interface 450 also includes a transfer-activation element 472 and a cancel element 474. Selection of the transfer-activation element 472 causes the data transfer to be performed. Selection of the cancel element 474 causes the data transfer to be canceled and may cause the display of the transfer-settings interface 450 to removed from the GUI 404.

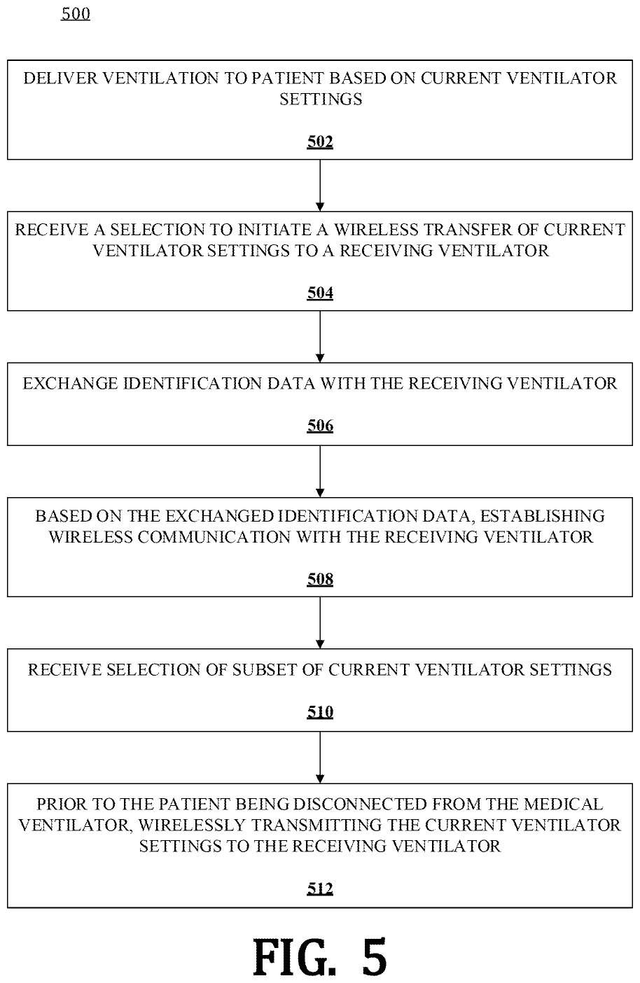

FIG. 5 depicts a flowchart illustrating an example method 500 for transferring ventilator settings. Method 500 may be performed by a ventilator, or components thereof, that is currently ventilating a patient that is to be transferred to a receiving ventilator. At operation 502, ventilation is delivered according to the current ventilator settings of the ventilator. At operation 504, a selection is received to initiate a wireless transfer of current ventilator settings to a receiving ventilator. Such a selection may be from a clinician via an interface of the ventilator.

Based on receiving the selection to initiate the wireless transfer of the current ventilator settings, identification data is exchanged with the receiving ventilator at operation 506. At operation 508, based on the exchanged identification data, communication with the receiving ventilator may be established. The communication may be wired or wireless and may be in the form of channel, session, etc. between the transferring ventilator and the receiving ventilator. For instance, operations 506 and 508 may be performed as part of a pairing process between the transferring ventilator and the receiving ventilator. As a result, after operation 508, the transferring ventilator and the receiving ventilator may be paired.

At operation 510, a selection of a subset of the current ventilator settings may be received. For instance, the ventilator may display an interface, such as transfer settings interface 408 in FIG. 4A, to receive the selection of the subset of current ventilator settings. The selection may also be received via such an interface.

At operation 512, the current ventilator settings are wirelessly transmitted, via the wireless communication established in operation 508, to the receiving ventilator. The current ventilator settings may include all the currently ventilator settings or a subset thereof, such as the subset of ventilator settings selection in operation 510. Operation 512 may also be performed prior to the patient being connected from the transferring ventilator that performs the operations of method 500.

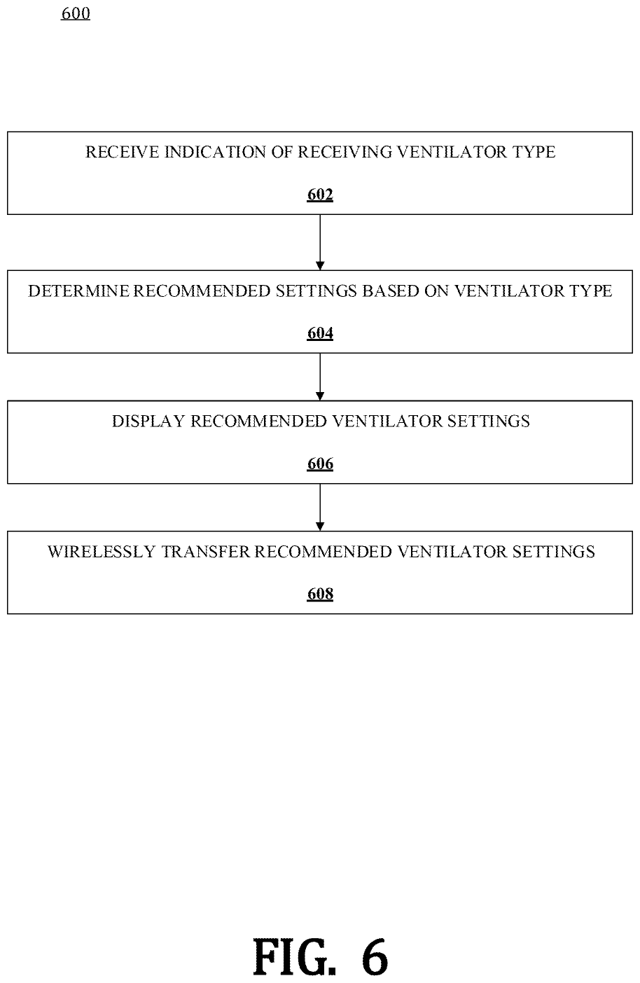

FIG. 6 depicts a flowchart illustrating an example method 600 for wirelessly transferring ventilator settings. Method 600 may be performed by a ventilator, or components thereof, that is currently ventilating a patient that is to be transferred to a receiving ventilator. Method 600 may also be performed as part of method 500 of FIG. 5.

At operation 602, an indication of the ventilator type of the receiving ventilator is received. The indication may be received as user input via an interface. For instance, the indication of the ventilator type may be received via a receiving ventilator section 415 of a transfer settings prompt 414, as depicted in FIG. 4B. In other examples, the indication of the ventilator type may be received from the receiving ventilator as identification data.

At operation 606, recommended ventilator settings are displayed on a display of the ventilator. The recommended ventilator settings may be ventilator settings to transfer to the receiving ventilator to cause the receiving ventilator to deliver ventilation that is substantially close to the ventilation being delivered by the transferring ventilator. A clinician may review the recommended settings, which may also include a recommended or closest mode, and determine whether to transfer those recommended settings. Upon confirmation or approval being received from the clinician, the recommended ventilator settings are transferred to the receiving ventilator at operation 608.

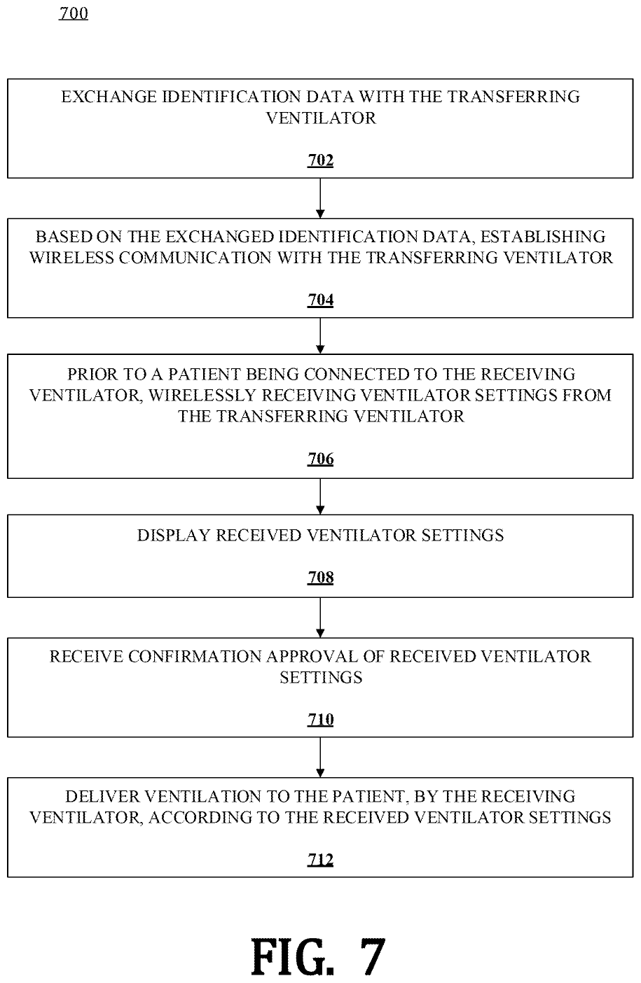

FIG. 7 depicts a flowchart illustrating an example method 700 for wirelessly receiving ventilator settings. Method 700 may be performed by a ventilator, or components thereof, that will be ventilating a patient after the patient is transferred from the transferring ventilator. At operation 702, identification data is exchanged with the transferring ventilator. At operation 704, based on the exchanged identification data, wireless communication is established with the transferring ventilator. Operations 702 and 704 may be substantially similar to operations 506 and 508 of method 500 in FIG. 5.

At operation 706, ventilator settings are wirelessly received from the transferring ventilator. The ventilator settings correspond to settings used by the transferring ventilator to provide ventilation to the patient. The ventilator settings may be received prior to the patient being connected to the receiving ventilator. Accordingly, the receiving ventilator may be ready to deliver ventilation according to the received ventilator settings as soon as the patient is connected.

At operation 708, the received ventilator settings are displayed on a display of the receiving ventilator. At operation 710, a confirmation approval of the received ventilator settings is received. The display of the received ventilator settings in operation 708 and the receipt of the confirmation approval may be achieved through a received-settings prompt 430 shown in FIG. 4D. Upon receiving the confirmation approval in operation 710, the received ventilator settings may be implemented. At operation 712, once the patient is connected to the receiving ventilator, the receiving ventilator delivers ventilation to the patient according to the received ventilator settings.

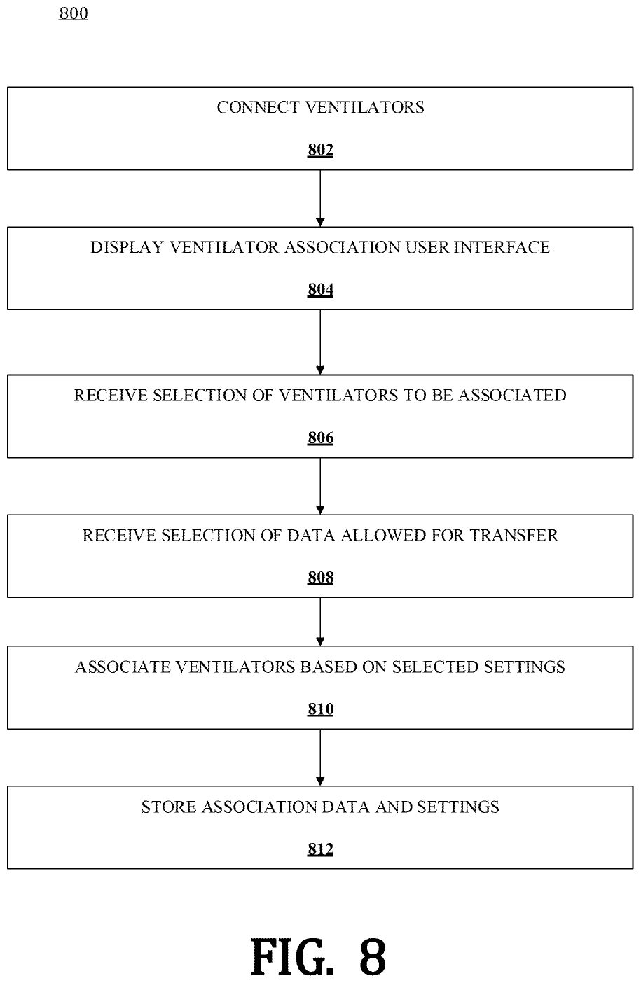

FIG. 8 depicts a method 800 for associating ventilators with one another. Method 800 may be performed to pre-authorize pairing or association of multiple ventilators. At operation 802, two or more ventilators are connected to one another either through a wireless connection (e.g., Bluetooth, Wifi, Wifi-Direct, Zigbee, Li-Fi, etc.) or a wired connection (e.g., TCP/IP, USB, etc.). At operation 804, a ventilator-association UI is displayed on one or more of the connected ventilators. The ventilator-association UI provides a listing of the connected ventilators. For instance, each of the ventilators may have a unique identifier (e.g., a universally unique identifier (UUID)). That unique identifier for each ventilator may be displayed in the UI. The unique identifier for each connected ventilator may also be displayed on the UI of the respective ventilator such that a user can see which physical ventilator corresponds to the unique identifier.

At operation 806, a selection is received of the ventilators that are to be associated with one another. Such a selection may be received through the UI as a selection of the unique identifiers of the connected ventilators. At operation 808, a selection of data that is allowed to be transferred between the selected ventilators is received. For instance, the UI may present different types of data that may be available for transfer between the ventilators. The types of data available for transfer may differ based on the type of ventilator. The user may then select the types of data that are allowed or authorized to be transferred.

At operation 810, the selected ventilators are associated with one another according to the settings and selections received in operations 806 and 808. Associating the ventilators with one another may include authorization operations such as a security certificate check. For example, each ventilator may be issued a security certificate from a certificate authority, such as a root node. This security certificate may be stored when the ventilator is manufactured and/or during an update. When an association between ventilators is attempted in operation 810, a check to ensure that the ventilators all have a trusted security certificate may be performed. If the ventilators do have a trusted security certificate, the association between the ventilators may be achieved.

At operation 812, the association data and selected settings (e.g., from operation 808) are stored in the associated ventilators. Where the association is a pairing, such as a Bluetooth pairing, the pairing information is also stored in the corresponding ventilators. By storing the pairing information, the ventilators may automatically be paired or wirelessly connected when the ventilators are brought into proximity with one another (e.g., within the Bluetooth range). Then, due to the pre-association of the ventilators, data may be more efficiently transferred at a later time while also allowed for additional layers of security to have already been accomplished. For instance, when a Bluetooth beacon of an associated ventilator is detected by one of the other associated ventilators, a communication channel may be automatically established between the two ventilators.

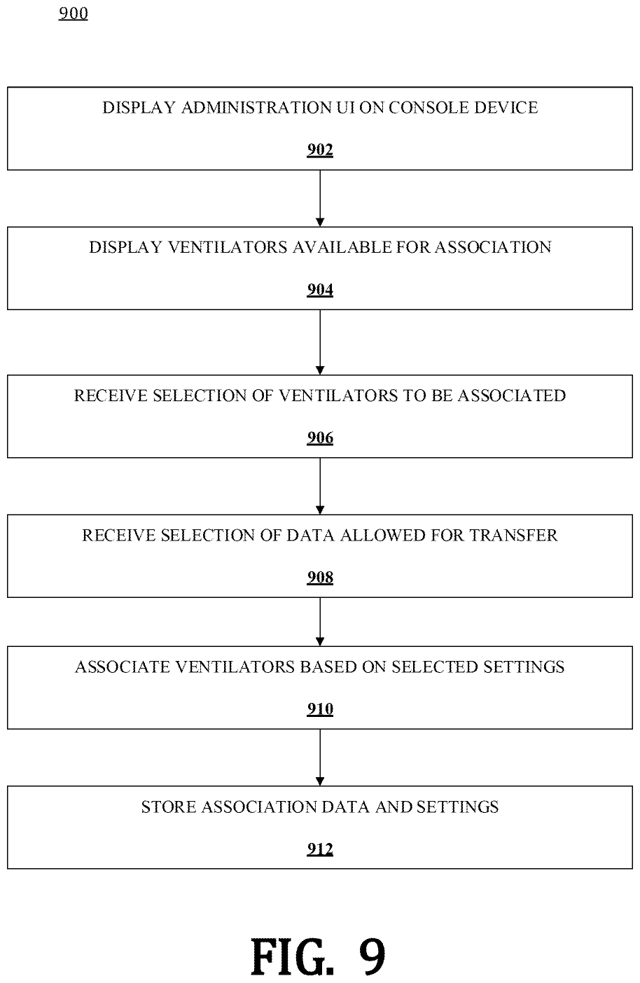

FIG. 9 depicts another method 900 for associating ventilators with one another. Similar to method 800, method 900 may be performed to pre-authorize pairing or association of multiple ventilators. At operation 902, an administration UI is displayed on console device, such as a laptop, smart phone, personal computer, etc. The administration UI may be accessed from a web browser or application with an Internet connection and the operations of the administration UI may be hosted by a server. The console device may be remotely located from the ventilators that are to be paired or associated. In other examples, the administration UI may be accessed and displayed on a UI of one of the ventilators.

At operation 904, the ventilators available for association are displayed in the administration UI. The ventilators may be displayed based on their unique ID and/or other identifying characteristics, such as their name and/or location, among other identifying data. The ventilators that are displayed may be specific to the user that is logged into the console or administration UI. For example, the displayed ventilators may include only those that are in the hospital, facility, and/or department associated with the current user.

At operation 906, a selection of the ventilators to be associated or paired is received. The selection of ventilators may be received as a drag-and-drop input or other types of events. At operation 908, a selection of data that is allowed to be transferred is received to indicate what data may be later transferred between the corresponding ventilators. At operation 910, the selected ventilators are associated or paired, and at operation 912 the association data and settings are stored on the respective ventilators. Operation 908-912 may be substantially similar to operations 808-812 discussed above.

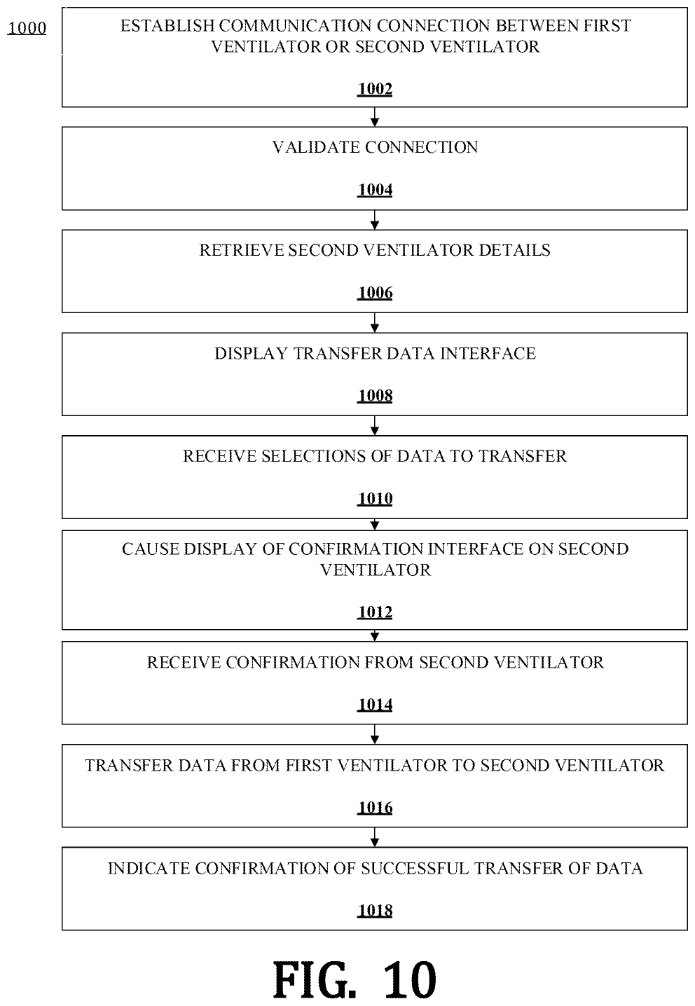

FIG. 10 depicts another method 1000 for transferring data from one ventilator to another ventilator. In operation 1002, a communication connection is established between a first ventilator and a second ventilator. The communication may be established as a wired or wireless connection. For instance, the communication may be established by connecting a cable between the ventilators. In wireless examples, the ventilators may have been previously associated or paired, such as via method 800 or 900 discussed above, and when the ventilators are brought into proximity of one another, a communication channel is automatically established between the first ventilator and the second ventilator over Bluetooth, Wifi, Wifi Direct, or other wireless protocols.

At operation 1004, an association or pairing between the first ventilator and the second ventilator is validated. Validation of the connection may include a check or validation of security certificates and/or a prior approval of an association between the two ventilators. For example, the first ventilator may check to see if the unique identifier for the second ventilator has been stored in the first ventilator as an authorized ventilator with which data can be transferred. The validation of the association may occur on the first ventilator and/or the second ventilator.

At operation 1006, details about the second ventilator are received. Such details may include the type of ventilator and/or the types of data or capabilities that are available for the second ventilator. In some examples, additional details about the state of the ventilator are also received. For instance, whether a patient circuit check (e.g., leak check) has been performed or other state information that indicates whether the second ventilator is ready to be connected to a patient and provide ventilation. Other state data may include use data, last ventilation start time, duration of ventilation, connection status, patient set up, patient circuit, attached peripherals, etc. Such state data may then be displayed on the first ventilator to provide the clinician an indication of when or whether the patient should be transferred to the second ventilator.

At operation 1008, a data transfer interface is displayed on the first ventilator. The data transfer interface may be similar to the interfaces described above, such as the interface depicted in FIG. 4F. For example, the data transfer interface may provide selectable elements for the data that is to be transferred to the second ventilator. At operation 1008, selections of data to transfer is received from the user, and a confirmation to transfer the selected data may also be received.

Based on the attempt to transfer data from the first ventilator, a signal may be sent from the first ventilator to the second ventilator to cause the second ventilator to display a confirmation interface at operation 1012. The confirmation interface may include a prompt for a user to confirm that data is to be received on the second ventilator. In some examples, a code may need to be input into the confirmation prompt. For instance, the first ventilator may present a code that needs to be entered in the second ventilator to confirm that the second ventilator is the intended target of the data. Such a confirmation process avoids unintentionally sending data to an unintended ventilator. At operation 1014, a confirmation is received from the second ventilator that indicates the second ventilator is ready to receive the data from the first ventilator. Such a confirmation signal may be generated from a user interaction with the confirmation prompt displayed on the second ventilator.

Based on receiving the confirmation signal from the second ventilator, the first ventilator then transfers the selected data at operation 1016. The data may first be encrypted prior to being sent to the second ventilator. As part of the association process, the second ventilator may be provided with a decryption key for decrypting the encrypted data. At operation 1018, once the data has been successfully transferred, the first ventilator and/or the second ventilator may indicate that the there has been a successful transfer of data. The indication of successful transfer may be audible or visual. An abort function may also be provided that allows for the data transfer to be aborted or canceled. Such an option may also be available after the transfer of data where selection of the abort option reverts the receiving ventilator to a prior state and/or removes the data that was transferred to the ventilator. The data that is transferred between the ventilators may also be logged or otherwise stored for later inspection.

Those skilled in the art will recognize that the methods and systems of the present disclosure may be implemented in many manners and as such are not to be limited by the foregoing aspects and examples. In other words, functional elements being performed by a single or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible.

Functionality may also be, in whole or in part, distributed among multiple components, in manners now known or to become known. Thus, a myriad of software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter. In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A method for transferring ventilator settings from one medical ventilator to another medical ventilator, the method comprising:
receiving a selection to initiate a transfer of current ventilator settings to a receiving ventilator, the current ventilator settings corresponding to settings currently being used by the medical ventilator to provide ventilation to a patient;
exchanging identification data with the receiving ventilator;
based on the exchanged identification data, establishing communication with the receiving ventilator; and
prior to the patient being disconnected from the medical ventilator, transmitting the current ventilator settings to the receiving ventilator.

2. The method of claim 1, further comprising:
receiving a selection of a subset of the current ventilator settings for transfer to the receiving ventilator; and
wherein transmitting the current ventilator settings includes transmitting the selected subset of the current ventilator settings.

3. The method of claim 1, further comprising:
displaying a transfer settings interface including a setting-selection menu and a transfer activation element;
receiving, via the setting-selection menu, a selection of a subset of ventilator settings for transfer to the receiving ventilator;
receiving a selection of the transfer activation element; and
based on receiving the selection of the transfer activation element, initiating the transmission, wherein transmitting the ventilator settings includes transmitting the selected subset of ventilator settings.

4. The method of claim 1, further comprising:
receiving an indication of a ventilator type of the receiving ventilator;
based on the ventilator type of the receiving ventilator and the current ventilator settings, generating recommended ventilator settings; and
wherein the transmitted current ventilator settings are the recommended ventilator settings.

5. The method of claim 4, wherein the indication of the ventilator type is based on input received via a user interface presented by the medical ventilator.

6. The method of claim 4, wherein the indication of the ventilator type is based on identification data received from the receiving ventilator.

7. The method of claim 1, further comprising transmitting, to the receiving ventilator, patient data for the patient.

8. A medical ventilator for transferring ventilator settings, the ventilator comprising:
a display;
a processor; and
memory storing instructions that, when executed by the processor, cause the medical ventilator to perform a set of operations comprising:
delivering ventilation to a patient based on current ventilator settings;
receiving a selection to initiate a transfer of the current ventilator settings to a receiving ventilator;
exchanging identification data with the receiving ventilator;
based on the exchanged identification data, establishing communication with the receiving ventilator; and
prior to the patient being disconnected from the medical ventilator, transmitting the current ventilator settings to the receiving ventilator.

9. The ventilator of claim 8, wherein the operations further comprise:
receiving a selection of a subset of the current ventilator settings for transfer to the receiving ventilator; and
wherein transmitting the current ventilator settings includes transmitting selected subset of the current ventilator settings.

10. The ventilator of claim 8, wherein the operations further comprise:
displaying, on the display, a transfer settings interface including a setting-selection menu and a transfer activation element;
receiving, via the setting-selection menu, a selection of a subset of ventilator settings for transfer to the receiving ventilator;
receiving a selection of the transfer activation element; and
based on receiving the selection of the transfer activation element, initiating the transmission, wherein transmitting the ventilator settings includes transmitting the selected subset of ventilator settings.

11. The ventilator of claim 8, wherein the operations further comprise:
receiving an indication of a ventilator type of the receiving ventilator;
based on the ventilator type of the receiving ventilator and the current ventilator settings, generating recommended ventilator settings; and
wherein the transmitted current ventilator settings are the recommended ventilator settings.

12. The ventilator of claim 11, wherein the indication of the ventilator type is based on input received via a user interface presented by the medical ventilator.

13. The ventilator of claim 11, wherein the indication of the ventilator type is based on identification data received from the receiving ventilator.

14. The ventilator of claim 8, wherein the operations further comprise transmitting, to the receiving ventilator, patient data for the patient.

15. A method for receiving ventilator settings from a transferring ventilator by a receiving ventilator, the method comprising:
exchanging identification data with the transferring ventilator;
based on the exchanged identification data, establishing communication with the transferring ventilator;
prior to a patient being connected to the receiving ventilator, receiving, by the receiving ventilator, ventilator settings from the transferring ventilator; and
delivering ventilation to the patient, by the receiving ventilator, according to the received ventilator settings.

16. The method of claim 15, further comprising displaying, by the receiving ventilator prior to the patient being connected, the received ventilator settings.

17. The method of claim 16, further comprising receiving a confirmation approval of the received ventilator settings, and wherein delivering ventilation is commenced based on receiving the confirmation approval.

18. The method of claim 15, further comprising:
receiving a selection to change the received ventilator settings;
displaying an interface to change the ventilator settings; and
receiving, via the interface, changes to the ventilator settings.

19. The method of claim 15, further comprising receiving, from the transferring ventilator, patient data for the patient being ventilated on the transferring ventilator.

20. The method of claim 19, further comprising based on the received patient data, accessing previously stored ventilator settings for the patient.
